(19) United States
(12) Patent Application Publication
(10) Pub. No.: US 2025/0352588 A1
(43) Pub. Date: Nov. 20, 2025

(54) EFFECT OF A PROBIOTIC COMPOSITION ON VAGINAL LACTOBACILLUS SPECIES

(71) Applicant: INTERNATIONAL N&H DENMARK APS, Kongens Lyngby (DK)

(72) Inventors: Mehreen Anjum, Espoo (FI); Liisa Lehtoranta, Sundsberg (FI); Markku Saarinen, Kantvik (FI); Pia Johanna M. Maukonen, Espoo (FI)

(21) Appl. No.: 18/696,298

(22) PCT Filed: Oct. 3, 2022
(86) PCT No.: PCT/EP2022/077463

§ 371 Date: Mar. 27, 2024

(30) Foreign Application Priority Data
Oct. 5, 2021 (EP) 21200985.6

(51) Int. Cl. A61K 35/747; C12N 1/20; C12R 1/225

(52) U.S. Cl. CPC A61K 35/747; C12N 1/205; C12R 2001/225

(57) ABSTRACT

This invention relates to compositions comprising an effective amount of bacterial strains of the genera Lacticaseibacillus and Lactobacillus as well as methods and uses of said compositions for promoting growth and metabolism of vaginal Lactobacillus species in a subject in need thereof.

### FIELD OF THE INVENTION

This invention relates to a new use of a probiotic composition comprising strains of the genera Lacticaseibacillus and Lactobacillus. The invention also relates to the use of said composition for promoting growth and metabolism of vaginal Lactobacillus species in a subject in need thereof. The invention further relates to a method of promoting growth and metabolism of vaginal Lactobacillus species in a subject in need thereof, comprising administering to said subject a composition comprising an effective amount of bacterial strains of the genera Lacticaseibacillus and Lactobacillus.

### BACKGROUND OF THE INVENTION

The healthy vaginal microbiota of women is primarily dominated by lactobacilli species more specifically single species belonging to either Lactobacillus crispatus, Lactobacillus iners, Lactobacillus gasseri, or Lactobacillus jensenii (Ravel et al., 2011). These lactic acid producing species are a characteristic of vaginal eubiosis. Lactobacillus spp. have been shown to have anti-pathogenic activity which is mainly attributed to their lactic acid secretion (O'Hanlon et al., 2011 Selle et al., 2013). Research shows that Lactobacillus crispatus dominated vaginal microbiota are the most stable (Gajer et al., 2012). In healthy women the acidic pH of the vaginal environment is maintained primarily by lactic acid (O'Hanlon et al., 2013). At physiological concentrations (e.g. 110 mM) at pH 4.5, lactic acid has shown to have anti-pathogenic effect against multiple bacterial vaginosis (BV) associated bacteria without decreasing the viability of the healthy vaginal lactobacilli isolates in vitro (O'Hanlon et al., 2011).

In contrast, the presence of diverse bacteria species and very few or no lactobacilli are characteristic of vaginal dysbiosis (Ling et al., 2010). The dysbiotic vaginal metabolic signature consists of vaginal pH higher than 4.5 and organic acid metabolite profile where lactic acid is decreased from physiological concentration of ˜110 mM to <20 mM and corresponding increase in short chain fatty acids (SCFAs) such as acetic acid and succinic acid, as well as an increase in biogenic amines (Aldunate et al., 2015; Gajer et al., 2012). The biogenic amines have been shown to destabilize the vaginal Lactobacillus spp. and play a role in reducing their protective effect on the vaginal environment by decreasing the lactic acid production (Borgogna et al., 2021).

Traditionally, probiotic lactobacilli have been selected for vaginal health based on their antimicrobial properties, i.e., their ability to produce antimicrobial compounds such as H₂O₂, bacteriocins, and their adhesion capability to vaginal epithelial cells which may inhibit attachment of pathogenic bacteria. Additionally, lactobacilli present the ability to acidify the vagina with lactic acid, immunomodulatory properties, and ability to survive and transiently colonize the vaginal epithelia (Tachedjian et al., 2017).

Some studies have investigated some bacterial species, such as Lacticaseibacillus rhamnosus, Limosilactobacillus reuteri, Lactobacillus acidophilus, Levilactobacillus brevis, Lactiplantibacillus plantarum, Lactobacillus gasseri and Lactobacillus crispatus, for their role in reducing the risk of bacterial vaginosis (BV) (Senok et al., 2009; Borges et al., 2014; Li et al., 2019).

Furthermore, several clinical studies have shown that Lacticaseibacillus rhamnosus HN001 (HN001) and Lactobacillus acidophilus La-14 (La-14) are beneficial in promoting overall the maintenance of healthy vaginal microbiota (De Alberti et al., 2015; Russo et al., 2018), reducing the risk for bacterial vaginosis (BV) (Russo et al., 2019a) and vulvovaginal candidiasis (Russo et al., 2019b). Furthermore, several preclinical trials show the antibacterial effect of HN001 and La-14 against pathogenic bacteria associated with vaginal pathogens (e.g. Inturri et al., 2016; Bertuccini et al., 2017; 2018; Jang et al., 2017).

However, to our knowledge the specific role of any probiotic strains in directly facilitating the growth of healthy vaginal species/isolates, e.g., Lactobacillus crispatus or Lactobacillus gasseri, or increasing the production of organic acids beneficial for vaginal health has not been investigated. As probiotics are generally marketed for healthy population, the information how specific probiotic strains interact with the members of the healthy vaginal community and potentially promote their abundance would be valuable when targeting these strains for the maintenance of healthy vaginal microbiota and reduction of infection risk.

### OBJECT OF THE INVENTION

The object of the present invention is to promote the growth and metabolism of vaginal lactobacilli isolates associated with healthy vaginal environment/microbiota in conditions mimicking physiological vaginal environment with specific probiotic combination.

In particular, the objective of the present invention is: 1) to provide growth advantage to vaginal lactobacilli characteristic in healthy women; 2) to promote the production/concentration of metabolites attributed to vaginal health from the vaginal Lactobacillus isolates; 3) to provide protection to vaginal isolates against the inhibitory effects of biogenic amines that are most commonly observed in the vaginal environment of healthy women with diverse vaginal microbiota characteristic of dysbiosis, and 4) to restore and improve the lactic acid production from strains of vaginal lactobacilli in the presence of biogenic amines.

### SUMMARY OF THE INVENTION

The present invention is based on the experiments described herein which demonstrate that strains of the species Lacticaseibacillus rhamnosus and Lactobacillus acidophilus promote the growth and metabolism of vaginal Lactobacillus spp. associated with healthy vaginal environment and microbiota. The present invention further demonstrates that the strains of Lacticaseibacillus rhamnosus and Lactobacillus acidophilus can provide protection against the harmful effects of biogenic amines in women.

Accordingly, in one aspect, the invention relates to a composition comprising an effective amount of bacterial strains of the genera Lacticaseibacillus and Lactobacillus for use in promoting growth and metabolism of vaginal Lactobacillus species in a subject in need thereof.

In another aspect, the invention relates to a use of a composition comprising an effective amount of bacterial strains of the genera Lacticaseibacillus and Lactobacillus for promoting growth and metabolism of vaginal Lactobacillus species in a subject in need thereof.

In a further aspect, the present invention relates to a method of promoting growth and metabolism of vaginal Lactobacillus species in a subject in need thereof, said method comprising administering to said subject a composition comprising an effective amount of bacterial strains of the genera Lacticaseibacillus and Lactobacillus.

### DESCRIPTION OF FIGURES

FIG. 1. Dual culture plate assay in simulated vaginal fluid (SVF) agar, pH 4.5. The Lactobacillus spp. growing on top of the probiotic spot (combination of strain HN001 and strain La-14) are visible. The figure shows an image of the section of probiotic spot visualized under a microscope at 10× magnification. The first image is of the probiotic spot with soft agar on top without Lactobacillus spp. The other images show the Lactobacillus spp. growing around and on the probiotic spot which is visible as the dark arc line in the images.

FIG. 2. Growth curves of a) Lactobacillus crispatus 13331, b) Lactobacillus crispatus 13348, c) Lactobacillus crispatus 20584, d) Lactobacillus gasseri 13224, e) Lactobacillus gasseri 20243 performed in SVF at pH 4.3±0.2 treated with supernatant (grey line) and control without supernatant (black). Optical density (y-axis) was determined for 48 hours (x-axis) at OD600 nm. The values are means from at least three individual experiments, each with three replicates. The significance was calculated using two-way ANOVA with Sidak's multiple comparison test. *P<0.05.

FIG. 3. The concentration (mM) (y-axis) of a) lactic acid, b) succinic acid and c) acetic acid was measured from the growth study samples after 48 h. The difference in concentration between Lactobacillus samples treated with and without (control) probiotic supernatant (PS) was measured using two-tailed paired t-tests. The values are means from three individual experiments; the error bars show the standard deviation. ns=non-significant, *P<0.05, **P<0.001, ***P<0.0001.

FIG. 4. Growth curves of Lactobacillus crispatus 13331 performed in SVF media at pH 4.3±0.2 with supernatant and biogenic amine (BA) (grey line), with BA (dotted line) and control without supernatant (black line). Optical density (y-axis) was determined for 44 hours (x-axis) at OD600 nm. The values are means from at least three individual experiments, each with three replicates. The significance was calculated using two-way ANOVA with Tukey's multiple comparison test *P<0.05.

FIG. 5. The mean lactic acid concentration (mM) (y-axis) from Lactobacillus crispatus 13331 after the growth experiment: strain alone (control) (black bar), with biogenic amine (BA) (light grey bar) and with probiotic supernatant (PS) and biogenic amine (dark grey bar). The x-axis shows the BA used in the experiment. The values are means from two individual experiments. The significance was calculated using two-way ANOVA with Tukey's multiple comparison test. *P<0.05.

### DETAILED DISCLOSURE OF THE INVENTION

The results as described in the present invention show that probiotic bacterial strains improve the growth and metabolism of vaginal Lactobacillus spp. The effect of probiotic bacterial strains on vaginal lactobacilli isolates was investigated in vitro in conditions simulating physiological environment of vagina.

The detailed aspects of this invention are set out below. In part some of the detailed aspects are discussed in separate sections. This is for ease of reference and is in no way limiting. All the embodiments described below are equally applicable to all aspects of the present invention unless the context specifically dictates otherwise.

### Bacteria

The bacterial strains of the present invention are selected from bacterial strains of the genera Lacticaseibacillus and Lactobacillus. Preferably the bacterial strains of the present invention are of the species Lactobacillus acidophilus, Lacticaseibacillus rhamnosus, Lactobacillus gasseri and Lactobacillus crispatus. In particular, the bacterial strains are chosen from Lactobacillus acidophilus strain La-14, Lacticaseibacillus rhamnosus strain HN001, Lactobacillus gasseri strain 13224 and Lactobacillus crispatus strain 13331 (Lc-122) and strain 13348. Strains La-14 and HN001 are commercially available from DuPont Nutrition Biosciences ApS.

The bacterial strains were also deposited by DuPont Nutrition Biosciences ApS, of Langebrogade 1, DK-1411 Copenhagen K, Denmark, in accordance with the Budapest Treaty at the Leibniz-Institut Deutsche Sammlung von Mikroorganismen und Zellkulturen GmbH (DSMZ), Inhoffenstrasse 7B, 38124 Braunschweig, Germany, where they are recorded under the following registration numbers:
1. Strain La-14 (DGCC11491); deposited on 1 Jun. 2021 under registration number DSM33880.
2. Strain HN001 (DGCC1460); deposited on 7 May 2021 under registration number DSM22876.
3. Strain 13331 (DGCC13331); deposited on 11 Aug. 2021 under registration number DSM33966.
4. Strain 13348 (DGCC13348); deposited on 11 Aug. 2021 under registration number DSM33968.
5. Strain 13224 (DGCC13224); deposited on 11 Aug. 2021 under registration number DSM33965.

Preferably the bacterial strains to be used in the present invention are bacterial strains which are generally recognised as safe and, which are preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

### Compositions

While it is possible to administer strains of the genera Lacticaseibacillus and Lactobacillus alone according to the present invention (i.e., without any support, diluent or excipient), said strains are typically administered on or in a support as part of a product, in particular as a component or at least as one of the components of a composition, a dietary supplement, a nutritional supplement, a food product or a pharmaceutical acceptable composition or formulation. These products typically contain additional components well known to those skilled in the art.

Therefore, in one embodiment, the present invention relates to a composition comprising an effective amount of bacterial strains of the genera Lacticaseibacillus and Lactobacillus for use in promoting growth and metabolism of vaginal Lactobacillus species in a subject in need thereof.

In a particular embodiment of the present invention, the bacterial strain of the genus Lacticaseibacillus is of the species Lacticaseibacillus rhamnosus. In another particular embodiment of the present invention, the bacterial strain of the genus Lactobacillus is of the species Lactobacillus acidophilus.

In another particular embodiment, the strain of the species Lacticaseibacillus rhamnosus is strain HN001. In another particular embodiment of the present invention the strain of the species Lactobacillus acidophilus is strain La-14.

In a particular embodiment to the present invention, the bacteria Lacticaseibacillus:Lactobacillus are present in the ratio of 1:4 (v/v).

In a further embodiment, the vaginal Lactobacillus species is Lactobacillus crispatus and or Lactobacillus gasseri.

In another embodiment, the vaginal Lactobacillus crispatus is strains 13331 (Lc-122) and/or strain 13348.

In a further embodiment, the vaginal Lactobacillus gasseri is strain 13224.

In another particular embodiment, the bacteria present in the composition according to the present invention are live bacteria.

The composition according to the present invention can be presented in different forms, such as a food product, food ingredient, a dietary supplement or a pharmaceutical acceptable composition or formulation.

In a further embodiment, the composition according to the present invention is encapsulated.

In another embodiment, the present invention relates to a method of promoting growth and metabolism of vaginal Lactobacillus species in a subject in need thereof, said method comprising administering to said subject a composition comprising an effective amount of bacterial strains of the genera Lacticaseibacillus and Lactobacillus.

In a particular embodiment of the present invention, the bacterial strain of the genus Lacticaseibacillus is of the species Lacticaseibacillus rhamnosus. In another particular embodiment of the present invention, the bacterial strain of the genus Lactobacillus is of the species Lactobacillus acidophilus.

In another particular embodiment, the strain of the species Lacticaseibacillus rhamnosus is strain HN001. In another particular embodiment of the present invention the strain of the species Lactobacillus acidophilus is strain La-14.

In a particular particular embodiment according to the present invention, the bacteria Lacticaseibacillus:Lactobacillus are present in the ratio of 1:4 (v/v).

In a further embodiment, the vaginal Lactobacillus species is Lactobacillus crispatus and or Lactobacillus gasseri.

In another embodiment, the vaginal Lactobacillus crispatus is strains 13331 (Lc-122) and/or strain 13348.

In a further embodiment, the vaginal Lactobacillus gasseri is strain 13224.

In another particular embodiment, the bacteria present in the composition used in the method according to the present invention are live bacteria.

The composition used in the method according to the present invention can be presented in different forms, such as a food product, food ingredient, a dietary supplement or a pharmaceutical acceptable composition or formulation.

In a particular embodiment of the present invention, the method of promoting growth and metabolism of vaginal Lactobacillus species in a subject in need thereof comprises a treatment regimen by oral administration of the composition.

In a particular embodiment, treatment regimen comprises vaginal administration of the composition.

In another particular embodiment, the treatment regimen comprises administering the composition for at least 1 week. In another embodiment, the treatment regimen comprises administering the composition for at least 2 weeks. In another embodiment, the treatment regimen comprises administering the composition at least two times per week. 31+32+33

In a further embodiment, the composition used in the method according to the present invention is encapsulated.

### Dosage

The bacterial strains used in accordance with the present invention may be present from 10⁶ to 10¹⁴ CFU of bacteria/g of support, and more particularly from 10⁸ to 10¹² CFU of bacteria/g of support, preferably 10⁹ to 10¹² CFU/g of support. By “support” is meant a composition, a food product, a food ingredient, a dietary supplement or a pharmaceutically acceptable composition.

Suitably, the bacterial strains of the genera Lacticaseibacillus and Lactobacillus used in accordance with the present invention may be administered at a dosage of from about 10⁶ to about 10¹⁴ CFU of microorganism/dose, preferably about 10⁸ to about 10¹² CFU of microorganism/dose and more preferably from about 10⁹ to about 10¹¹ CFU of microorganism/dose. CFU stands for “colony-forming units”. By the term “per dose” it is meant that this amount of microorganism is provided to a subject either per day or per intake, preferably per day. For example, if the microorganisms are to be administered in a food product, for example in a yoghurt, then the yoghurt will preferably contain from about 10⁸ to 10¹² CFU of the microorganism. Alternatively, however, this amount of microorganism may be split into 5 multiple administrations each consisting of a smaller amount of microbial loading-so long as the overall amount of microorganism received by the subject in any specific time, for instance each 24-hour period, is from about 10⁶ to about 10¹² CFU of microorganism, preferably 10⁸ to about 10¹² CFU of microorganism and more preferably from about 10⁹ to about 10¹¹ CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, the composition according to the present invention comprises at least 1×10⁶ colony forming units (CFUs) of the bacterial strains of the genera Lacticaseibacillus and Lactobacillus.

### Food Product

In one embodiment, the bacterial strains are used according to the invention in a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

When used as, or in the preparation of, a food, such as functional food, the bacteria of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the bacteria of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, vegetable milk, acidified soy/juice beverage, aseptic/retorted chocolate drink, bar mixes, beverage powders, calcium fortified soy/plain and chocolate milk, calcium fortified coffee beverage.

Advantageously, where the product is a food product, the bacterial strains should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment age.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Dietary Supplements

The compositions of the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help prevent or treat diseases.

### Medical Food

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Composition

The bacteria of the present invention may be used as—or in the preparation of—a pharmaceutical composition or formulation. Here, the term “pharmaceutical” is used in a broad sense—and covers pharmaceuticals for humans as well as pharmaceuticals for animals (i.e. veterinary applications).

In a preferred embodiment, the pharmaceutical acceptable composition is a medicament.

The pharmaceutical composition can be for therapeutic purposes-which may be curative or palliative or preventative in nature.

In a preferred embodiment of the present invention, the medicament is for oral administration.

A pharmaceutically acceptable composition or support may be for example a formulation or support in the form of creams, foams, gels, lotions, and ointments of compressed tablets, tablets, capsules, ointments, suppositories or drinkable solutions.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid-depending on the use and/or the mode of application and/or the mode of administration.

The bacterial strains of the present invention may be used as pharmaceutical ingredients. Here, the composition may be the sole active component, or it may be at least one of a number (i.e. 2 or more) of active components.

The bacterial strains may be used according to the present invention in any suitable form-whether when alone or when present in a combination with other components or ingredients. Likewise, combinations comprising the bacteria of the present invention and other components and/or ingredients (i.e. ingredients-such as food ingredients, functional food ingredients or pharmaceutical ingredients) may be used in any suitable form.

The bacterial strains may be used according to the present invention in the form of solid or liquid preparations or alternatives thereof. Examples of solid preparations include, but are not limited to tablets, capsules, dusts, granules and powders which may be water dispersible, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the bacteria of the present invention are used in a tablet form-such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid 30 monoglycerides and diglycerides, petroethrai fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the bacteria of the present invention may be combined with various sweetening or flavouring agents, colouring matter or dyes, with emulsifying and/or suspending agents and with diluents such as water, propylene glycol and glycerin, and combinations thereof.

The forms may also include gelatine capsules; fibre capsules, fibre tablets etc.; or even fibre beverages.

In one aspect, the bacteria according to the present invention may be administered in an aerosol, for example by way of a nasal spray, for instance for administration to the respiratory tract.

### Prebiotics

In one embodiment, the bacterial strains and compositions of the present invention may further be combined or comprise one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal or skin microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat and unrefined barley.

Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, (human) milk oligosaccharides and all forms of resistant starches.

The combination of one or more of the bacterial strains according to the present invention and one or more fibres and/or prebiotics according to the present invention exhibits a synergistic effect in certain applications (i.e. an effect which is greater than the additive effect of the bacteria when used separately).

In one embodiment, the bacterial strains or a mixture thereof according to the present invention is used in combination with one or more fibres and/or prebiotic.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, microcrystalline cellulose (MCC), milk oligosaccharide or betaine.

In a further aspect, the invention relates to a composition, food products, food ingredient, dietary supplements or a pharmaceutical acceptable composition comprising bacterial strains according to the present invention or a mixture thereof and one or more fibres and/or a prebiotic.

### EMBODIMENTS OF THE INVENTION

For the avoidance of doubt, some of the embodiments the present invention relates to are set out below:
Embodiment 1. Composition comprising an effective amount of bacterial strains of the genera Lacticaseibacillus and Lactobacillus for use in promoting growth and metabolism of vaginal Lactobacillus species in a subject in need thereof.
Embodiment 2. The composition for use according to embodiment, wherein the bacterial strain of the genus Lacticaseibacillus is of the species Lacticaseibacillus rhamnosus.
Embodiment 3. The composition for use according to embodiment 1, wherein the bacterial strain of the genus Lactobacillus is of the species Lactobacillus acidophilus.
Embodiment 4. The composition for use according to embodiment 2, wherein the strain of the species Lacticaseibacillus rhamnosus is strain HN001.
Embodiment 5. The composition for use according to embodiment 3, wherein the strain of the species Lactobacillus acidophilus is strain La-14.
Embodiment 6. The composition for use according to any one of the embodiments 1-5, wherein the bacteria Lacticaseibacillus:Lactobacillus are present in the ratio of 1:4 (v/v).
Embodiment 7. The composition for use according to any one of embodiments 1-6, wherein the vaginal Lactobacillus species is Lactobacillus crispatus and or Lactobacillus gasseri.
Embodiment 8. The composition for use according to embodiment 7, wherein Lactobacillus crispatus is strains 13331 (Lc-122) and/or strain 13348.
Embodiment 9. The composition for use according to embodiment 7, wherein Lactobacillus gasseri is strain 13224.
Embodiment 10. The composition for use according to any one of the embodiments 1-9, wherein said composition is a food product, a food ingredient, a dietary supplement or a pharmaceutical acceptable composition.
Embodiment 11. Use of a composition comprising an effective amount of bacterial strains of the genera Lacticaseibacillus and Lactobacillus for promoting growth and metabolism of vaginal Lactobacillus species in a subject in need thereof.
Embodiment 12. The use according to embodiment 11, wherein the bacterial strain of the genus Lacticaseibacillus is of the species Lacticaseibacillus rhamnosus.
Embodiment 13. The use according to embodiment 11, wherein the bacterial strain of the genus Lactobacillus is of the species Lactobacillus acidophilus.
Embodiment 14. The use according to embodiment 12, wherein the strain of the species Lacticaseibacillus rhamnosus is strain HN001.
Embodiment 15. The use according to embodiment 13, wherein the strain of the species Lactobacillus acidophilus is strain La-14.
Embodiment 16. The use according to any one of the embodiments 11-15, wherein the bacteria Lacticaseibacillus:Lactobacillus are present in the ratio of 1:4 (v/v).
Embodiment 17. The use according to any one of embodiments 11-16, wherein the vaginal Lactobacillus species is Lactobacillus crispatus and or Lactobacillus gasseri.
Embodiment 18. The use according to embodiment 17, wherein Lactobacillus crispatus is strains 13331 (Lc-122) and/or strain 13348.
Embodiment 19. The use according to embodiment 17, wherein Lactobacillus gasseri is strain 13224.
Embodiment 20. A method of promoting growth and metabolism of vaginal Lactobacillus species in a subject in need thereof, said method comprising administering to said subject a composition comprising an effective amount of bacterial strains of the genera Lacticaseibacillus and Lactobacillus.
Embodiment 21. The method according to embodiment 20, wherein the bacterial strain of the genus Lacticaseibacillus is of the species Lacticaseibacillus rhamnosus.
Embodiment 22. The method according to embodiment 20, wherein the bacterial strain of the genus Lactobacillus is of the species Lactobacillus acidophilus.
Embodiment 23. The method according to embodiment 21, wherein the strain of the species Lacticaseibacillus rhamnosus is strain HN001.
Embodiment 24. The method according to embodiment 22, wherein the strain of the species Lactobacillus acidophilus is strain La-14.
Embodiment 25. The method according to any one of the embodiments 20-24, wherein the bacteria Lacticaseibacillus:Lactobacillus are present in the ratio of 1:4 (v/v).
Embodiment 26. The method according to any one of embodiments 20-25, wherein the vaginal Lactobacillus species is Lactobacillus crispatus and or Lactobacillus gasseri.
Embodiment 27. The method according to embodiment 26, wherein Lactobacillus crispatus is strains 13331 (Lc-122) and/or strain 13348.
Embodiment 28. The method according to embodiment 26, wherein Lactobacillus gasseri is strain 13224.
Embodiment 29. The method according to any one of embodiments 20-29, wherein said method comprises a treatment regimen by oral administration of the composition.
Embodiment 30. The method according to embodiment 29, wherein the treatment regimen comprises vaginal administration of the composition.
Embodiment 31. The method according to embodiment 29, wherein the treatment regimen comprises administering the composition for at least 1 week.
Embodiment 32. The method according to any one of embodiments 29, wherein the treatment regimen comprises administering the composition for at least 2 weeks.
Embodiment 33. The method according to any one of embodiments 29-32, wherein the treatment regimen comprises administering the composition at least two times per week.
Embodiment 34. The method according to any one of embodiments 20-33, wherein said composition comprises at least 1×10⁶ colony forming units (CFUs) of the bacterial strains of the genera Lacticaseibacillus and Lactobacillus.
Embodiment 35. The method according to any one of embodiments 20-34, wherein the composition is encapsulated.

### EXAMPLES

The following examples are provided to demonstrate and further illustrate specific embodiments and aspects of the present invention and are not to be construed as limiting the scope thereof.

### Materials and Methods

### Strains and Growth Conditions

The Lactobacillus strains investigated in this study are listed in Table 1. The probiotic strains Lactobacillus acidophilus La-14 and Lacticaseibacillus rhamnosus HN001 were obtained from Danisco Global Culture Collection (DGCC). Lactobacillus crispatus 13331, 13348 and Lactobacillus gasseri 13224 are human vaginal isolates that were obtained from DGCC. Lactobacillus crispatus 20584 isolated from human eye and the human isolate Lactobacillus gasseri 20243 were obtained from Deutsche Sammlung von Mikroorganismen und Zellkulturen (DSMZ). The non-vaginal isolate Lactobacillus crispatus 20584 and Lactobacillus gasseri 20243 served as control strains to compare if the probiotic supernatant would affect the vaginal and non-vaginal isolates of same species differently. The anoxic conditions were produced using mixed gas from Anoxomat® III anaerobic culture system (80% N₂, 10% CO₂, 10% H₂) for all experiments throughout the study. The experiments were performed in the simulated vaginal fluid (SVF) at 37° C. for 24 h or 48 h under anoxic conditions. The SVF was prepared fresh for each experiment according to the recipe (Pan et al., 2020). The pH of the media was adjusted to 4.3±0.2 by lactic acid at final concentration 88 mM. The media was pre-reduced by adding 0.05% (w/v) L-cysteine hydrochloride and stored at 4° C. until needed.

The lactobacilli isolates were adapted to SVF pH 4.5 (Brandt and Borgogna et al., 2020), by inoculating them first in de Man, Rogosa, and Sharpe (MRS) or Bifidobacterium growth media 58 (Salli et al., 2021) from frozen stocks that were stored at −80° C., depending on their nutrient requirement, and were incubated at 37° C. for 24 h under anoxic conditions. The next day, strains were passaged in SVF, pH 4.5 at 1% of total media volume and incubated again at 37° C. for up to 48 h. The sub-passage was repeated once more in SVF pH 4.5 before the strains were used for the experiments. For each set of experiments the strains were adapted to the SVF media independently.

The probiotic combination was made by mixing the strains HN001 and La-14 in 1:4 ratio (v/v) (Jang et al., 2017) for all experiments.

TABLE 1
Lactobacilli strains investigated in this study

| Species | Strain | Culture collection | Media | Isolation source |
|---|---|---|---|---|
| Lactobacillus | 13331 | DGCC | MRS | Human vagina |
| crispatus | (Lc-122) | | | |
| Lactobacillus | 13348 | DGCC | MRS | Human vagina |
| crispatus | | | | |
| Lactobacillus | 20584 | DSMZ | Bifidobacterium | Human eye |
| crispatus | | (DSM20584) | medium 58 | |
| Lactobacillus | 13224 | DGCC | MRS | Human vagina |
| gasseri | | | | swab |
| Lactobacillus | 20243 | DSMZ | MRS | Human |
| gasseri | | (DSM20243) | | |
| Lacticaseibacillus | 1460 | DGCC | MRS | Dairy |
| rhamnosus | (HN001) | | | |
| Lactobacillus | 11491 | DGCC | MRS | Human intestine |
| acidophilus | (La-14) | | | |

DGCC: Danisco Global Culture Collection.
DSMZ: Deutsche Sammlung von Mikroorganismen und Zellkulturen.

### Dual-Culture Plate Assay

Dual culture plate assay was performed to determine the effect of probiotic combination of HN001 and La-14 on Lactobacillus spp. growth on agar plate when in contact with live probiotic culture. The SVF agar plates were prepared by supplementing the SVF media pH 4.5 with 1.5% agar. The lactobacilli strains including the probiotics were inoculated in SVF media pH 4.5 and incubated at 37° C. under anoxic conditions. The required ratio of the probiotic combination HN001 and La-14 was obtained by mixing the overnight cultures in the ratio 1:4 (v/v), respectively prior to the experiment.

Two spots of volume 10 μl from probiotic mix were placed on SVF agar plates at an equal distance and allowed to dry for ˜1 h. Soft agar tubes 7 ml each were freshly prepared on the day of the experiment using SVF media supplemented with 0.7% of agar. The sterile soft agar tubes were kept in water bath at 45° C. until further use.

The overnight cultures of lactobacilli strains were diluted 100-fold by adding 70 μl of bacteria culture to 7 ml of sterile soft agar in a test tube. The tube was vortexed, and the soft agar was poured in the middle of the agar plate with probiotic spots and allowed to spread evenly. The agar was allowed to solidify before the plates were incubated anoxically at 37° C. for 48 h. The colonies of lactobacilli strains growing around and over the spot were visualized under a microscope (Leica Microsystems GmbH, Germany).

### Preparation of Cell Free Supernatant of Probiotic Combination

Supernatant was prepared from the probiotic strains HN001 and La-14. The probiotic strains were inoculated individually in SVF from frozen (−80° C.) stocks and incubated at 37° C. for 24 h. Next day, the strains were sub-cultured in the SVF media and incubated at 37° C. for 24 h. The pH for each probiotic strain after overnight growth was ˜4.3±0.2. The probiotic cultures HN001 and La-14 were mixed in 1:4 v/v, respectively, in a 50 ml conical centrifuge tube (Falcon tube). The culture mix was spun down at 10 000× g for 20 minutes. The supernatant was carefully separated and frozen at −20° C.

### Growth Experiments Using Probiotic Supernatant

Bacterial growth experiments were performed in SVF pH 4.5 using Bioscreen© C system (Labsystems). The growth experiments were performed as described previously with some modifications (Mäkeläinen et al., 2010).

The lactobacilli strains were inoculated from frozen (−80° C.) stocks and adapted to pH 4.5 in SVF media as mentioned above and incubated at 37° C. for 24 h. Bacterial culture dilutions (1000 fold) were prepared in SVF media pH 4.5. The bacterial dilution was added at 10% (20 μl) of total volume (200 μl) in each well thus resulting in the final concentration of bacteria approximately 10\hat{ }5 CFU/ml per well. Probiotic supernatant pH 4.5, was added at 10% concentration (20 μl) of the total well volume (200 μl) to the required wells on the honey-comb plate. The remaining volume in each well was made up to 200 μl with the SVF media. The growth of lactobacilli strains, treated with and without (control) the probiotic supernatant in growth media, was monitored. Each strain with or without of the supernatant was added in triplicates to the honey-comb plate.

Bacterial growth was monitored by measuring the optical density at 600 nm every 30 min for 48 h at 37° C. in a Bioscreen reader placed in an anaerobic cabinet. Growth curves for each strain were compared with or without the presence of the supernatant. Growth experiments were performed in at least three biological replicates. After 48 h the samples from the growth experiment were pooled from the triplicate wells and spun down at 10,000×g for 15 min. The supernatant was carefully removed and frozen at −20° C. These samples were further analyzed for the change in selected metabolite concentrations in the presence of probiotic supernatant during the growth study.

Growth Experiments with Probiotic Supernatant and Biogenic Amines

The potential growth inhibitory effects of biogenic amines cadaverine, putrescine, tyramine, spermine and spermidine were tested against vaginal isolate of Lactobacillus crispatus 13331. The growth experiment was performed as described above but with some modifications. The Lactobacillus crispatus strain was cultured in SVF media (control), and in the SVF media containing one of the biogenic amines, and in the SVF media containing one of the biogenic amines and probiotic supernatant. The effect of all five biogenic amines was investigated independently on the pure culture of Lactobacillus crispatus 13331. Each test condition was investigated in triplicates in the honey-comb plate. The biogenic amines were added at the highest physiological concentration of cadaverine (4990 μM), putrescine (3179 μM), tyramine (813 μM), spermine (186 μM) and spermidine (59 μM) measured from vaginal samples in a previous study (Borgogna et al., 2021). The growth study was performed in SVF pH 4.5 using Bioscreen© C system measuring the optical density at 600 nm every 30 min for 44 h at 37° C. The growth curves were compared for each test condition with its growth in the presence of biogenic amine and with biogenic amine including probiotic supernatant. Growth experiments were performed in three independent biological replicates.

After 44 h the samples from the growth experiment were pooled from the triplicate wells and centrifuged at 10,000×g for 15 min. The supernatant was carefully removed and frozen at −20° C. Two randomly selected sample sets were further analyzed for the change in lactic acid concentrations from L. crispatus 13331 alone, in the presence of biogenic amine and, with biogenic amine and probiotic supernatant.

### Analysis of Microbial Metabolites

Amino acids and succinic acid in bacterial fermentation samples were determined using the gas chromatographic-mass spectrometric method by McMillan et al. (2015) with modifications. The sample solution (50 μl) was mixed with the internal standard in methanol (200 μl 68 nM ribitol) and the protein in the sample was allowed to precipitate. After centrifugation (16000×g, 5 min), an aliquot (30 μl) of the supernatant was evaporated to dryness at 30° C. under a stream of nitrogen. Next, the analytes were derivatized first with methoxylamine hydrochloride (50 μl, 15 mg/ml in pyridine, 50° C. for 90 min) and then with N,O-Bis(trimethylsilyl)-trifluoroacetamide with 1% trimethylchlorosilane (BSTFA-1% TMCS 50 μl, 70° C. for 60 min) into volatile trimethylsilyl ether derivatives. The sample (1 μl) was injected into the gas chromatograph using pulsed splitless injection (splitless time 0.7 min, pulse flow 2 ml/min, pulse time 0.7 min). The analytes were separated on a HP-5 ms capillary column (30 m×0.25 mm id, phase thickness 0.25 μm, Agilent, Waldbronn, Germany) using helium as the carrier gas at the flow rate of 1 ml/min. Following temperature program was used for separation: Initial temperature 60° C. was hold for 2 min, then increased to 140° C. at the rate of 10° C./min, then increased to 240° C. at the rate of 4° C./min, and finally increased to 300° C. at the rate of 10° C./min and hold for 8 min. The analytes were detected with a mass selective detector and concentration of succinic acid, alanine, aspartic acid, isoleucine, leucine, phenylalanine, and tryptophan was calculated using the internal standardization method.

Lactic acid and acetic acid were analyzed in bacterial fermentation samples with gas chromatography, as described previously (Ouwehand et al. 2009) with modifications. An internal standard (100 μl 20 mM pivalic acid), 300 μl of water and 250 μl of saturated oxalic acid solution were added to 0.1 ml of sample. After thorough mixing, the sample was allowed to stand for 60 min at 4° C. and then centrifuged at 16 000× g for 5 min. Supernatant (1 μl) was analyzed by gas chromatography using a glass column packed with 80/120 Carbopack BDA/4% Carbowax 20 M stationary phase (2 m×2 mm, Supelco, Bellefonte PA, USA) at 175° C. and using helium as the carrier gas at a flow rate of 24 ml/min. The temperatures of the injector and the flame ionization detector were 200° C. and 245° C., respectively. The concentration of lactic acid and acetic was calculated using the internal standardization method.

### Statistical Analysis

In the growth experiments the optical density between lactobacilli strain growth versus the lactobacilli strain growth in the presence of probiotic supernatant were compared at each time point and statistical difference was measured by two-way ANOVA with Sidak's multiple comparison test. The change in concentration of metabolites after the growth study was analyzed by comparison between control and test group and the statistical differences was analyzed using two-tailed paired t-test.

The change in the growth of lactobacilli strains within the three groups: control, strain with biogenic amine and strain with biogenic amine and probiotic supernatant was analyzed using two-way ANOVA with Turkey's multiple comparison test. The change in concentration of lactic acid from the growth study with biogenic amines was measured using two-way ANOVA with Tukey's multiple comparison test. The statistical analysis was performed using GraphPad Prism version 9.1 for Windows (GraphPad Software). P values of <0.05 were considered to be significant.

### Results

The results showed that the bacterial strains mixture improved the growth of vaginal isolates significantly in their presence compared to growth of vaginal isolates alone. The metabolites important for vaginal health were also increased significantly from vaginal isolates in the presence of bacterial strains mixture. The bacterial strains provided protection to vaginal isolate from growth inhibitory properties of biogenic amines and also improved the metabolite levels that are important for vaginal health maintenance.

### The Vaginal Lactobacilli Showed Dense Growth on Live Cultures of Probiotics HN001 and La-14 Combination

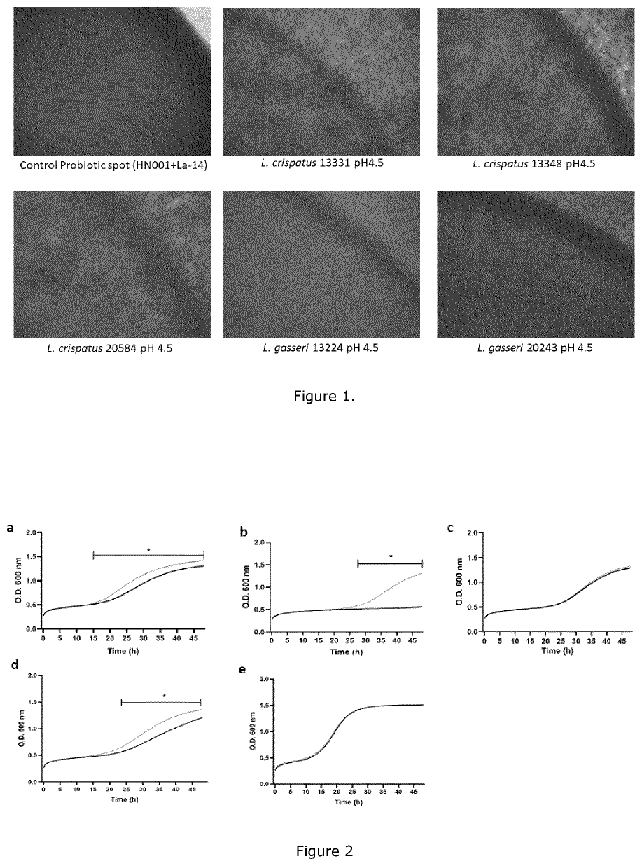

The dual culture plate assay in SVF showed that the Lactobacillus spp. investigated in this study grew densely on top of and around the probiotic combination of HN001 and La-14. FIG. 1 shows the growth of lactobacilli isolates visualized using the microscope at 10× magnification. Therefore, the probiotics do not inhibit the growth of these indigenous vaginal isolates of lactobacilli. Previous studies show that HN001 and La-14 have inhibitory effects on vaginal pathogens associated in BV (Gardenerella vaginalis and Atopobium vaginae) and aerobic vaginitis (Staphylococcus aureus and Escherichia coli) in vitro (Bertuccini et al., 2017) and in mice (Jang et al., 2017). This indicates that the probiotic combination interacts differently with commensal lactobacilli as opposed to pathogens in media simulating vaginal secretions.

### The Probiotics HN001 and La-14 Supernatant Promotes Growth of the Vaginal Lactobacilli Isolates

The optical density (OD) at 600 nm was used to monitor the growth of the lactobacilli strains with and without (control) the presence of probiotic supernatant. The results showed that the growth of L. crispatus 13331 with probiotic supernatant statistically significantly increased after 15 h and remained significantly higher than the growth of L. crispatus 13331 without probiotic supernatant until the end of the experiment (FIG. 2a). The strain L. crispatus 13348 showed significantly higher growth after 28 h in the presence of probiotic supernatant as compared to its own control (FIG. 2b). On the other hand, the non-vaginal isolate L. crispatus 20584 had no difference in growth with or without the probiotic supernatant (FIG. 2c).

L. gasseri 13224 strain showed significantly higher growth in the presence of probiotic supernatant after 24 h until the end of the experiment compared to its own control (FIG. 2d). Similar to the observation with L. crispatus strains, the non-vaginal isolate L. gasseri 20243 showed no difference in growth with or without the presence of the probiotic supernatant (FIG. 2e).

These observations indicate that the probiotic supernatant of HN001 and La-14 provides growth advantage specifically to vaginal isolates in the SVF media. It is also possible that since the vaginal isolates are more adapted to the vaginal environment (SVF media and low pH) than the non-vaginal isolates, the growth benefit from probiotic supernatant is more visible.

The production of lactic acid and succinic acid increases significantly in vaginal lactobacilli strains grown in the presence of probiotic supernatant

The concentration of lactic acid, succinic acid, and acetic acid was measured after the growth experiment from the lactobacilli strains with or without probiotic supernatant.

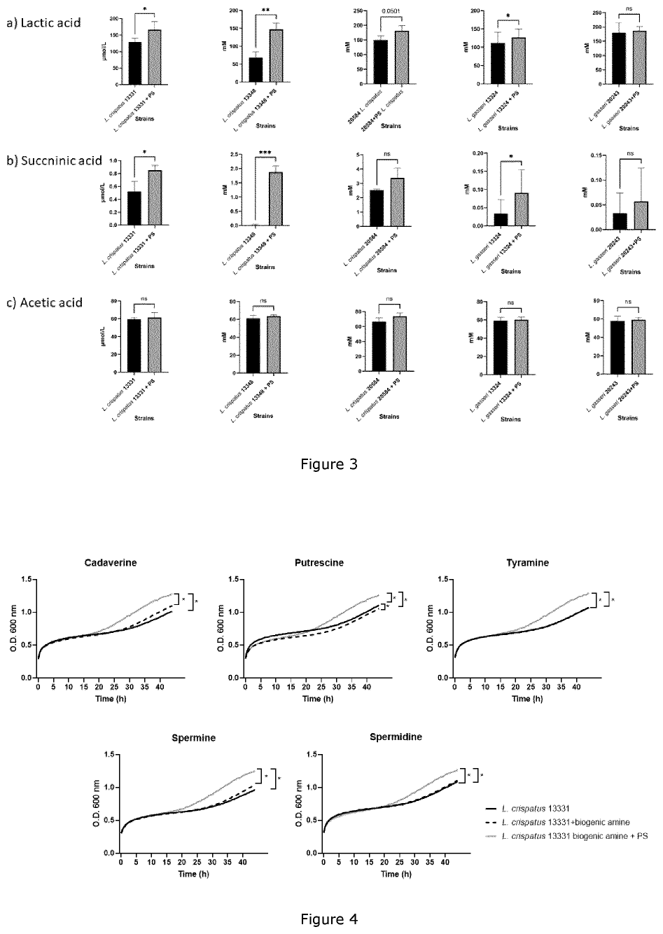

After 48 h, for L. crispatus 13331 the mean concentration of lactic acid was 129 mM but in the presence of probiotic supernatant the mean concentration was 166 mM, which was significantly higher (p=0.02) (FIG. 3a). Similarly, for L. crispatus 13348 the mean concentration of lactic acid was 68 mM without probiotic supernatant while in the presence of supernatant the mean concentration increased to 147 mM and the increase was statistically significant (p=0.001) (FIG. 3a). For L. crispatus 20584, the mean concentration of lactic acid was 150 mM without supernatant and 182 mM with supernatant, however this was not statistically significant (p=0.0501) (FIG. 3a). The mean concentration of lactic acid measured from L. gasseri 13224 without supernatant was 111 mM and with supernatant 127 mM, which was significantly higher (p=0.03) (FIG. 3a). The mean concentration of lactic acid measured from L. gasseri 20243 without supernatant was 180 mM and with supernatant 187 mM, which was not statistically significant difference (p=0.6) (FIG. 3a). The concentration of lactic acid was also measured from the probiotic supernatant in order to assess whether the observed increase in concentration was due to the supernatant itself. The concentration of lactic acid in the 10% probiotic supernatant added was 2.9 mM, which does not explain the increase observed for lactic acid concentration from the vaginal isolates of lactobacilli.

After 48 h, the mean concentration of succinic acid for the L. crispatus 13331 was 0.5 mM but in the presence of probiotic supernatant the mean concentration increased to 0.8 mM, which was significantly higher (p=0.01) (FIG. 3b). Similarly, for L. crispatus 13348 the mean concentration of succinic acid was 0.01 mM without probiotic supernatant while in the presence of supernatant the mean concentration increased to 1.9 mM and the increase was statistically significant (p=0.0005) (FIG. 3b). For L. crispatus 20584 the mean concentration of succinic acid was 2.5 mM without supernatant and 3.4 mM with supernatant, however this increase was not statistically significant (p=0.1) (FIG. 3b). The mean concentration of succinic acid measured from L. gasseri 13224 without supernatant was 0.03 mM and with supernatant was 0.09 mM, which was significantly higher (p=0.03) (FIG. 3b). The mean concentration of succinic acid measured from L. gasseri 20243 without supernatant was 0.03 mM and with supernatant was 0.06 mM, however, this increase was not statistically significant (p=0.2) (FIG. 3b). The concentration of succinic acid measured from the 10% probiotic supernatant was 0.008 mM.

The mean acetic acid concentration measured from L. crispatus 13331 was 59 mM without supernatant and 62 mM with supernatant. From L. crispatus 13348 the acetic acid concentration was 61 mM without supernatant and 64 mM with supernatant and from L. crispatus 20584 it was 67 mM without supernatant and 74 mM with supernatant. None of these observed increases were statistically significant (p=0.6, 0.2, 0.2 respectively) (FIG. 3c). From L. gasseri 13224 the mean acetic acid concentration measured was 59 mM without supernatant and 60 mM with supernatant and for L. gasseri 20243 the concentration was 58 mM without supernatant and 59 mM with supernatant which were not statistically significant (p=0.1 and 0.7 respectively) (FIG. 3c). The concentration of acetic acid measured from the 10% probiotic supernatant was 1.4 mM.

The probiotic combination of strains HN001 and La-14 showed protective effect on vaginal lactobacilli growth against all tested biogenic amines under physiological conditions

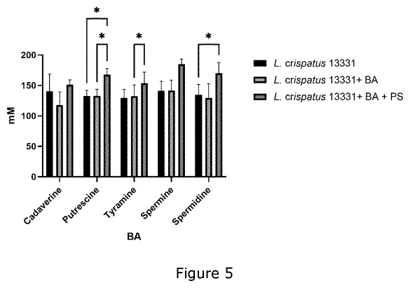

The vaginal isolate L. crispatus 13331 was selected as a model organism to determine the difference in growth due to the presence of biogenic amines and to assess the protective effect of the probiotic supernatant with the biogenic amine. There was no statistically significant difference in growth between the growth of L. crispatus 13331 alone (control) or in the presence of cadaverine. However, there was significant increase in the growth of L. crispatus 13331 with cadaverine in the presence of probiotic supernatant from 21.5 h until 43 h compared to L. crispatus 13331 alone (control). There was also statistically significant increase in the growth of L. crispatus 13331 in the presence of cadaverine and probiotic supernatant compared to L. crispatus 13331 with only cadaverine from 19.5 to 35.5 h (FIG. 4).

There was a significant reduction in the growth of L. crispatus 13331 in the presence of putrescine compared with the growth of L. crispatus 13331 (control) from 0.5 h to the end of the experiment. Putrescine has been shown to negatively affect the growth of L. crispatus strains in a recent study (Borgogna et al., 2021). The growth of L. crispatus 13331 with putrescine and probiotic supernatant was significantly higher from 1 h to 20.5 h and then later from 26 h to 39.5 h compared to the growth of L. crispatus 13331 alone. Additionally, the growth was significantly higher of L. crispatus 13331 with biogenic amine putrescine and probiotic supernatant from 16.5 h to 43 h when compared to L. crispatus 13331 with putrescine (FIG. 4).

In the test condition with tyramine there was a significant increase in the growth of L. crispatus 13331 in the presence of tyramine with probiotic supernatant compared to growth of the strain alone (control), from 20 h to 40.5 h. The growth of L. crispatus 13331 in the presence of tyramine with probiotic supernatant was also significantly higher from 25 h to the end of experiment compared to growth of L. crispatus 13331 with tyramine alone (FIG. 4).

In the test condition with spermine there was a significant increase in the growth of L. crispatus 13331 in the presence of spermine with probiotic supernatant from 19.5 h to the end of experiment compared to growth of the strain alone (control). The growth of L. crispatus 13331 in the presence of spermine with probiotic supernatant was also significantly higher from 19.5 h to 40.5 h compared to growth of 13331 with spermine alone (FIG. 4).

Lastly, in the test condition with spermidine the growth of L. crispatus 13331 was significantly higher in the presence of spermidine with probiotic supernatant from 24.5 h up to 39.5 h compared to strain alone. Also, the increase in L. crispatus 13331 growth was significantly higher with spermidine and probiotic supernatant 24.5 h to 37.5 compared to the growth of the strain with spermidine alone (FIG. 4).

The results imply that not only the probiotic supernatant provides growth advantage to L. crispatus 13331 as seen before, but the probiotic supernatant also provides additional growth advantage when the biogenic amines are present.

The probiotic combination HN001 and La-14 supernatant lead to increase in concentration of lactic acid by L. crispatus 13331 in the presence of tested biogenic amines

Recently, a study conducted by Borgogna et al. (2021) showed that the biogenic amines that are released mostly by vaginal pathogens during dysbiosis can cause decrease in lactic acid production from healthy lactobacilli isolates in the vaginal environment. This decrease allows the vaginal pathogens to survive even better as the decrease in lactic acid concentration leads to increase in pH. We measured the concentration of lactic acid from the vaginal isolate L. crispatus 13331 when the biogenic amines were present and compared it to the concentration of lactic acid when the probiotic supernatant was also present along with the biogenic amine.

Overall, the concentration of lactic acid was higher in all tested conditions where the probiotic supernatant was present regardless of the biogenic amine used (FIG. 5). In the test condition with cadaverine the mean lactic acid concentration from the strain alone was 141 mM and decreased to 118 mM in the presence of cadaverine, however, the supernatant increased the mean concentration 151 mM even in the presence of cadaverine. This change between all three test conditions was non-significant (FIG. 5).

In the test condition with putrescine the mean lactic acid concentration from the strain alone (control) and in the presence of putrescine was 133 mM, however, in the presence of probiotic supernatant the mean lactic acid concentration increased to 168 mM which was significantly higher compared to both control (p=0.02) and biogenic amine test condition (p=0.02) (FIG. 5).

In the test condition with tyramine the mean lactic acid concentration from the strain alone (control) was 129 mM and in the presence of tyramine increased to 132 mM, however, in the presence of probiotic supernatant the mean lactic acid concentration increased to 154 mM which was significantly higher compared to lactic acid levels from strain with biogenic amine (p=0.02) (FIG. 5).

In the test condition with spermine the mean concentration of lactic acid from both the strain alone (control) and in the presence of spermine was 141 mM which increased to 185 mM in the presence of probiotic supernatant with spermine. This increase was non-significant after multiple comparisons tests.

In the test condition with spermidine the mean concentration of lactic acid from the strain alone (control) was 135 mM which decreased to 129 mM in the presence of spermidine. In the presence of probiotic supernatant with spermidine the mean concentration of lactic acid increased to 170 mM. The increase was significantly higher in the presence of probiotic supernatant compared to the strain alone (p=0.02) (FIG. 5).

In conclusion, under physiological conditions the probiotic supernatant leads to increase in concentration of lactic acid produced by L. crispatus 13331 even in the presence of biogenic amine, which allows the maintenance of low pH characteristic of healthy vaginal environment.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Aldunate, M., Srbinovski, D., Hearps, A. C., Latham, C. F., Ramsland, P. A., Gugasyan, R., Cone, R. A., and Tachedjian, G. (2015). Antimicrobial and immune modulatory effects of lactic acid and short chain fatty acids produced by vaginal microbiota associated with eubiosis and bacterial vaginosis. Front Physiol 6, 164.
Bertuccini, L., Russo, R., Iosi, F., and Superti, F. (2017). Effects of Lactobacillus rhamnosus and Lactobacillus acidophilus on bacterial vaginal pathogens. Int J Immunopathol Pharmacol 30, 163-167.
Bertuccini, L., Russo, R., Iosi, F., and Superti, F. (2018). Lactobacilli and lactoferrin: Biotherapeutic effects for vaginal health. J Funct Foods 45, 86-94.
Borges, S., Silva, J., and Teixeira, P. (2014). The role of lactobacilli and probiotics in maintaining vaginal health. Arch Gynecol Obstet 289, 479-489.
Borgogna, J. C., Shardell, M. D., Grace, S. G., Santori, E. K., Americus, B., Li, Z., Ulanov, A., Forney, L., Nelson, T. M., Brotman, R. M., Ravel, J., Yeoman, C. J. (2021). Biogenic amines increase the odds of bacterial vaginosis and affect the growth of and lactic acid production by vaginal Lactobacillus spp. Appl Environ Microbiol. 87, e03068-20.
Brandt, K. and Barrangou, R. (2020). Adaptive response to iterative passages of five Lactobacillus species in simulated vaginal fluid. BMC Microbiology 20, 339.
De Alberti, D., Russo, R., Terruzzi, F., Nobile, V., and Ouwehand, A. C. (2015). Lactobacilli vaginal colonisation after oral consumption of Respecta ((R)) complex: a randomised controlled pilot study. Arch Gynecol Obstet 292, 861-867.
Gajer, P., Brotman, R. M., Bai, G., Sakamoto, J., Schutte, U. M., Zhong, ×., Koenig, S. S., Fu, L., Ma, Z. S., Zhou, X., et al. (2012). Temporal dynamics of the human vaginal microbiota. Sci Transl Med 4, 132ra152.
Hill, C., Guarner, F., Reid, G. et al. (2014) The International Scientific Association for Probiotics and Prebiotics consensus statement on the scope and appropriate use of the term probiotic. Nat Rev Gastroenterol Hepatol 11, 506-514.
Inturri, R., Stivala, A., Furneri, P. M., Blandino, G. (2016). Growth and adhesion to HT-29 cells inhibition of Gram-negatives by Bifidobacterium longum BB536 e Lactobacillus rhamnosus HN001 alone and in combination. Eur Rev Med Pharmacol Sci. 20, 4943-4949.
Jang, S. E., Jeong, J. J., Choi, S. Y., Kim, H., Han, M. J., and Kim, D. H. (2017). Lactobacillus rhamnosus HN001 and Lactobacillus acidophilus La-14 Attenuate Gardnerella vaginalis-Infected Bacterial Vaginosis in Mice. Nutrients 9.
Li, C., Wang, T., Li, Y., Zhang, T., Wang, Q., He, J., Wang, L., Li, L., Yang, N., Fang, Y. (2019). Probiotics for the treatment of women with bacterial vaginosis: A systematic review and meta-analysis of randomized clinical trials. Eur J Pharmacol. 864, 172660.
Ling, Z., Kong, J., Liu, F. Zhu, H. Chen, X. Wang, Y. Li, L. Nelson, K. E. Xia, Y. Xiang, C. (2010) Molecular analysis of the diversity of vaginal microbiota associated with bacterial vaginosis. BMC Genomics 11:488.
Mäkeläinen, H. S.m Saarinen, M., Stowell, J., Rautonen, N., and Ouwehand, A. C. (2010). Xylo-oligosaccharides and lactitol promote the growth of Bifidobacterium lactis and Lactobacillus species in pure cultures. Benef microbes. 1, 139-48.
McMillan, A., Rulisa, S., Sumarah, M., Macklaim, J. M., Renaud, J., Bisanz, J. E., Gloor, G. B., and Reid, G. (2015). A multi-platform metabolomics approach identifies highly specific biomarkers of bacterial diversity in the vagina of pregnant and nonpregnant women. Sci Reports. 5:14174.
O'Hanlon, D. E., Moench, T. R., and Cone, R. A. (2011). In vaginal fluid, bacteria associated with bacterial vaginosis can be suppressed with lactic acid but not hydrogen peroxide. BMC Infect Dis 11, 200.
O'Hanlon, D. E., Moench, T. R., and Cone, R. A. (2013). Vaginal pH and microbicidal lactic acid when lactobacilli dominate the microbiota. PLOS One 8, e80074.
Ouwehand A C, Tiihonen K, Saarinen M, Putaala H., and Rautonen N, 2009. Influence of a combination of Lactobacillus acidophilus NCFM and lactitol on healthy elderly: intestinal and immune parameters. Br J Nutr 101:367-375.
Pan, M., Hidalgo-Cantabrana, C., Goh, Y. J., Sanozky-Dawes, R., and Barrangou, R. (2020). Comparative analysis of Lactobacillus gasseri and Lactobacillus crispatus isolated from human urogenital and gastrointestinal tracts. Front Microbiol 10, 3146.
Ravel, J., Gajer, P., Abdo, Z., Schneider, G. M., Koenig, S. S., McCulle, S. L., Karlebach, S., Gorle, R., Russell, J., Tacket, C. O., et al. (2011). Vaginal microbiome of reproductive-age women. Proc Natl Acad Sci USA 108 Suppl 1, 4680-4687.
Russo, R., Edu, A., and De Seta, F. (2018). Study on the effects of an oral lactobacilli and lactoferrin complex in women with intermediate vaginal microbiota. Arch Gynecol Obstet 298, 139-145.
Russo, R., Karadja, E., and De Seta, F. (2019a). Evidence-based mixture containing Lactobacillus strains and lactoferrin to prevent recurrent bacterial vaginosis: a double blind, placebo controlled, randomised clinical trial. Benef Microbes 10, 19-26.
Russo, R., Superti, F., Karadja, E., and De Seta, F. (2019b). Randomised clinical trial in women with Recurrent Vulvovaginal Candidiasis: Efficacy of probiotics and lactoferrin as maintenance treatment. Mycoses 62, 328-335.
Salli, K., Hirvonen, J., Siitonen, J., Ahonen, I., Anglenius, H., and Maukonen, J. (2021). Selective utilization of the human milk oligosaccharides 2′-Fucosyllactose, 3-Fucosyllactose, and difucosyllactose by various probiotic and pathogenic bacteria. J. Agric. Food Chem. 69, 170-182.
Selle, K., and Klaenhammer, T. R. (2013). Genomic and phenotypic evidence for probiotic influences of Lactobacillus gasseri on human health. FEMS Microbiol Rev 37, 915-935.
Senok, A. C., Verstraelen, H., Temmerman, M., and Botta, G. A. (2009). Probiotics for the treatment of bacterial vaginosis. Cochrane Database Syst Rev, CD006289.
Tachedjian, G., Aldunate, M., Bradshaw, C. S., and Cone, R. A. (2017). The role of lactic acid production by probiotic Lactobacillus species in vaginal health. Res Microbiol 168, 782-792.

1. A method of promoting growth and metabolism of vaginal Lactobacillus species in a subject in need thereof comprising administering comprising an effective amount of a composition comprising bacterial strains of the genera Lacticaseibacillus and Lactobacillus for use in promoting growth and metabolism of vaginal Lactobacillus species in to a subject in need thereof.

2. The method of claim 1, wherein the bacterial strain of the genus Lacticaseibacillus is of the species Lacticaseibacillus rhamnosus.

3. The method of claim 1, wherein the bacterial strain of the genus Lactobacillus is of the species Lactobacillus acidophilus.

4. The method of claim 2, wherein the strain of the species Lacticaseibacillus rhamnosus is strain HN001.

5. The method of claim 3, wherein the strain of the species Lactobacillus acidophilus is strain La-14.

6. The method of claim 1, wherein the bacteria Lacticaseibacillus:Lactobacillus are present in the ratio of 1:4 (v/v).

7. The method of claim 1, wherein the vaginal Lactobacillus species is Lactobacillus crispatus and or Lactobacillus gasseri.

8. The method of claim 7, wherein the Lactobacillus crispatus is strains 13331 (Lc-122) and/or strain 13348.

9. The method of claim 7, wherein Lactobacillus gasseri is strain 13224.

10. The method of claim 1, wherein the composition is a food product, a food ingredient, a dietary supplement or a pharmaceutical acceptable composition.

11-15. (canceled)
